(19) United States
(12) Patent Application Publication
(10) Pub. No.: US 2005/0250725 A1
(43) Pub. Date: Nov. 10, 2005

(54) Regulation of the P21 gene and uses thereof

(76) Inventors: Peter M. Price, Little Rock, AR (US); Judit Megyesi, Little Rock, AR (US); Robert Safirstein, Little Rock, AR (US)

Correspondence Address: Benjamin Aaron Adler;ADLER & ASSOCIATES, 8011 Candle Lane, Houston, TX, 77071, US

(21) Appl. No.: 11/112,354

(22) Filed: Apr. 22, 2005

Related U.S. Application Data
(63) Continuation-in-part of application No. 09/881,635, filed on Jun. 14, 2001.
(60) Provisional application No. 60/212,224, filed on Jun. 16, 2000.

(51) Int. Cl. A61K 48/00; A61K 31/19

(52) U.S. Cl. 514/44; 514/569; 514/557; 514/575

(57) ABSTRACT

The present invention in general is directed to methods for manipulating cyclin-dependent kinase inhibitor activity. Such manipulation will reduce or eliminate the expression of p21 gene based on specific requirements. This will be helpful in treating or preventing pathophysiological state in an individual which is characterized by undesirable level of cyclin-dependent kinase inhibitor activity, treating chronic progessive renal failure or lowering the rate of long-term rejection of a transplated organ in an individual. Additionally, the present invention also teaches up-regulating the expression of p21 gene using histone deacetylase inhibitors. The use of such inhibitors alone will be be helpful in treating acute renal failures whereas in combination with chemotherapeutic drugs will enhance the therapeutic potential of such drugs.

### CROSS-REFERENCE TO RELATED APPLICATION

This is a continuation-in-part of non-provisional application U.S. Ser. No. 09/881,635, filed Jun. 14, 2001, which claims benefit of provisional application U.S. Ser. No. 60/212,224, filed Jun. 15, 2000, now abandoned.

### FEDERAL FUNDING LEGEND

This invention was produced in part using funds obtained through grants R01 DK54471 and PO1 DK58324 from the National Institutes of Health. Consequently, the federal government has certain rights in this invention.

### BACKGROUND OF THE INVENTION

1. Field of the Invention

The present invention relates generally to the fields of molecular biology, nephrology, oncology, and organ transplantation. More specifically, the present invention relates to methods of regulating expression of the p21 gene to reduce the progression of chronic renal disease by reducing p21 expression, to reduce the severity of acute renal failure by upregulating the p21 gene, and to improve susceptibility of cancer cells to chemotherapeutics and to reduce concomitant renal failure during the chemotherapy.

2. Description of the Related Art

The removal of substantial amounts of renal tissue is followed by a progressive decline in renal function (1,2). Glomerular hypertrophy occurs early in response to this ablation and is accompanied by short-term increases in glomerular filtration (3,4). These structural and functional adaptations to loss of excretory function are thought to be maladaptive and to influence the progression to end stage renal disease. Progression is initially seen as localized increases in mesangial matrix that then leads to global glomerular sclerosis, and is usually associated with systemic hypertension, which has been speculated to accelerate its course. Although the early glomerular hypertrophy and hyperfunction, especially the glomerular hypertension that determines it, have been invoked as predeterminants of the later destructive effects of renal ablation, there is no established causal link between these events and the progressive nature of the renal disease.

Acute short-term stress in the kidney provokes molecular responses that involve the expression of several genes, including the cyclin-dependent kinase (cdk) inhibitor p21 (5). p21 plays a critical role in processes by which nuclear events subsequent to environmental stress are regulated. p21 is induced to very high levels by oxidative stress (6) and DNA damage (7). The p21 protein (8) acts as an inhibitor of cyclin-dependent kinase activity (9) and effectively stops cell-cycle progression (8,9). p21 is over expressed in many cells undergoing senescence (10) or terminal differentiation (11,12). The expression of p21 following short term chemotoxic renal stress is rapid and expression of p21 under these circumstances played a protective role (13). Chronic, long term stress could provoke sustained expression of p21 and that such expression could influence renal function and morphology. Thus, controlling p21 function may ameliorate or even prevent progressive end-stage renal disease or other pathophysiological states in other organs.

Histone deacetylase (HDAC) inhibitors, which are undergoing clinical trials in cancer therapy (32), have differential affects in cancer cells as opposed to normal cells. While histone deacetylase inhibitors are cytoprotective in normal cells, they provoke cell death and sensitize cancer cells to chemotherapeutic agents. It was observed that the p21 cyclin-dependent kinase inhibitor, which was upregulated by histone deacetylase inhibitors (33), was cytoprotective in kidney cells exposed to cisplatin. Furthermore, it was observed that p21 KO cells were more sensitive to cisplatin and that up-regulation of p21 by gene transfer was protective (13,34).

Thus, although the histone deacetylase inhibitors caused death of cancer cells and sensitized the cancer cells to additional cytotoxic agents, the outcome in cancer and normal cells was directly related to p21 up-regulation. It is further speculated that the difference in the outcome may be due to a dysregulation of the cell cycle in cancer cells. Hence, the p21 gene product was crucial in proper regulation of the cell cycle. Lung cancer represents an attractive model for studying the differential regulation of cell cycle in normal and cancer cells. Although cisplatin is the cornerstone of medical therapy for both small-cell, and non-small-cell lung carcinoma, the status of cell cycle control is not similar in these subtypes. In small cell cancers, the retinoblastoma (RB) gene is deleted in the overwhelming majority. Accordingly, the ultimate substrate of p21/CDK2/CyclinE axis (RB) is missing, thus providing a good control system to dissect the effect of p21 regulation, and contrast it with non-small cell lung cancer (NSCLC) (where retinoblastoma function is present—albeit not at fully normal levels).

The prior art is deficient in the lack of gene regulation to treat chronic organ failure and cancer. Additionally, it lacks the understanding of the relationship between cytotoxicity and the regulation of p21 gene. The present invention fulfills this long-standing need and desire in the art.

### SUMMARY OF THE INVENTION

The present invention demonstrated a probable mechanism for the differential effect of cell cycle regulation in normal and cancer cells. The present invention relates to the exploitation of this difference to enhance the clinical usefulness of histone deacetylase inhibitors. The present invention is directed to a method for treating or preventing a pathophysiological state of an organ in an individual, where the state is characterizd by an undesirable level of cyclin-dependent kinase inhibitor activity in the organ. This method comprises the step of regulating the expression of the p21 gene in the organ of the individual.

The present invention also is directed to a method for treating or preventing chonic progressive renal failure in the individual. This method comprises the step pf regulating the expression of the p21 gene in one or both kidneys of the individual, where the regulation of the p21 gene results in the manipulation of cyclin-dependent kinase inhibitor activity in one or both kidneys.

The present invention is further directed to a method of lowering the rate of long-term rejection of a transplanted organ in an individual. This method comprises the step of transplanting into the individual the organ from a donor where the p21 gene in the organ is not expressed.

The present invention is still further directed to a method of enhancing therapeutic potential of a chemotherapeutic drug in an individual being treated for a pathophysiological state. This method comprises administering an histone deacetylase inhibitor to the individual and sensitizing target cells to the chemotherapeutic drug and protecting normal cells from the drug-induced cytotoxicity, thereby enhancing the therapeutic potential of the chemotherapeutic drug in the individual.

The present invention is also directed to a method of treating or preventing acute renal failure in an individual. This method comprises the step of administering an histone deacetylase inhibitor to the individual to upregulate the p21 gene briefly to reduce the severity of the renal failue.

The present invention is further directed to a method of treating a pathophysiological state in an individual. This method comprises administering an histone deacetylase inhibitor to the individual. Such an administration upregulates the expression of cell cycle regulator gene which induces cell cycle arrest, differentiation or apoptosis in damaged cells, thereby treating the pathophysiological state in the individual.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows renal function following ablation. Clearance of inulin (ml per minute) is calculated per gram kidney is calculated in mice from both genotypes. Statistically significant differences are only noted between the two populations at 14-16 weeks after ablation (p=0.04). Values shown in the figure represent±standard error.

FIG. 2 shows the mean arterial pressure. Mean systolic blood pressure is obtained by catheterizing the left femoral artery. Statistically significant differences between the two populations is noted as early as 6-8 weeks after ablation (p=0.005), which increases by 14-16 weeks after ablation (p=0.00002). Values represent±standard error.

FIGS. 3A-3F show the histologic changes in remnant kidney after ablation. Representative sections are untreated (FIGS. 3A and 3B), 8 weeks (FIGS. 3C and 3D), 16 weeks (FIG. 3E), or 26 weeks (FIG. 3F) after ablation of wild-type (FIGS. 3A, 3C, and 3E) or p21(−/−) mice (FIGS. 3B, 3D, and 3F). Sections are stained with periodic acid-Schiff (PAS). Magnification is ×390.

FIGS. 4A-4B show the detection of interstitial fibrosis using trichrome stain in remnant kidney after ablation. Representative sections are 6 weeks (FIG. 4A) or 26 weeks (FIG. 4B) after ablation of wild-type (FIG. 4A) or p21(−/−) mice (FIG. 4B) Magnification is ×390.

FIGS. 5A-5B show the in situ hybridization for localization of p21 mRNA in remnant kidney cells after partial renal ablation. Hybridization of an antisense p21 probe to RNA in cells of remnant kidney is shown at 4 weeks (FIG. 5A) and 14 weeks (FIG. 5B) after ablation. Magnification is ×390.

FIGS. 6A-6B show the cell cycle analysis in remnant kidney cells after partial renal ablation. Immunodetection of nuclear PCNA localization is shown 2 weeks after ablation in kidney sections from p21(−/−) (FIG. 6A) and from wild-type mice (FIG. 6B). Magnification is ×390.

FIG. 7 shows the increase in renal p21 expression after ischemia-reperfusion, cisplatin administration, and during urinary tract obstruction.

FIG. 8 shows the increased susceptibility of the kidney to cisplatin in mice without the p21 gene. Blood Urea Nitrogen, which measures renal function, is retained to a higher degree indicating greater renal failure, in mice without the p21 gene.

FIGS. 9A-9H show the effect of the p21 gene in protecting renal proximal tubule cells from cisplatin-induced apoptosis and identifies the N-terminal portion of the protein as the protective part of the molecule. FIG. 9H also shows that inhibition of cdk2, the principal target of p21 also protects renal cells from damage.

FIGS. 10A-10D shows the effect of HDAC inhibitor in renal cells in the presence or absence of cisplatin and/or phenylbutyrate on the distribution of DNA. The X-axis displays intensity of propidium iodide flouresence and the y-axis is the number of events at each intensity level. Control cells show a normal distribution of DNA in untreated cells (FIG. 10A), while cells exposed to 50 uM cisplatin (FIG. 10B) show fragmented apoptotic distribution of DNA. Cells incubated with phenylbutyrate (PB, 100 uM) for twenty four hours alone (FIG. 10C) show the normal distribution of DNA, while those exposed to PB and cisplatin (FIG. 10D) show more normal distribution of DNA. Percent apoptosis is also indicated.

### DETAILED DESCRIPTION OF THE INVENTION

In one embodiment of the present invention there is provided a method for treating or preventing a pathophysiological state of an organ in an individual where the state is characterized by an undesirable level of cyclin-dependent kinase inhibitor acivity in the organ, comprising the step of regulating the expression of the p21 gene in the organ of the individual. Generally, the organ is selected from the group consisting of kidney, heart, liver and lung. The pathophysiological state is selected from the group consisting of renal fibrosis, glomerulosclerosis, reduced filtration rates, hypertension and organ transplantation rejection. Furthermore, the regulation of the expression of the p21 gene results in the reduction or elimination of the expression of the p21 gene. Additionally, the reduction or elimination of the expression of the p21 gene is performed by a technique selected from the group consisting of drug therapy and genetic manipulation.

In another embodiment of the present invention, there is provided a method for treating or preventing chronic progressive renal failure in an individual, comprising the step of regulating the expression of the p21 gene in one or both kidneys of the individual, where the regulation of the p21 gene results in the manipulation of cyclin-dependent kinase inhibitor activity in one or both kidneys. The regulation of the expression of p21 gene results in the reduction or elimination of the expression of the p21 gene. Generally, the reduction or elimination of the p21 gene is performed by a technique selected from the group consisting of drug therapy and genetic manipulation.

In yet another embodiment of the present invention, there is provided a method of lowering the rate of long-term rejection of a transplanted organ in an individual comprising the step of transplanting into the individual the organ from a donor where the p21 gene in the organ is not expressed. Generally, the organ is selected from the group consisting of kidney, heart, liver and lung.

In still yet another embodiment of the present invention, there is provided a method of enhancing therapeutic potential of a chemotherapeutic drug in an individual being treated for a pathophysiological state, comprising: administering a histone deacetylase (HDAC) inhibitor to the individual, sensitizing the target cells to the chemotherapeutic drug and protecting the normal cells from the drug-induced cytotoxicity, thereby enhancing the therapeutic potential of the chemotherapeutic drug in the individual. The administration of such an inhibitor results in upregulation of expression of the p21 gene. Furthermore, the normal cells are protected from drug-induced cytotoxicity not limited to but including nephrotoxicity, mucositis, enterocilitis or bone marrow suppression. Representative examples of histone deacetylase inhibitor include phenylbutyrate, suberoylamide (SAHA), valproic acid, and trichostatin and their derivatives and representative examples of the chemotherapeutic drug include cisplatin, all-trans retinoic acid, gemcitabine, and amphotericin. Generally, the individual is being treated for a pathophysiological state not limited to but including lung cancer, ovarian cancer, testicular cancer or head and neck cancer.

In another embodiment of the present invention, there is provided a method of treating or preventing acute renal failure in an individual, comprising the step of administering an histone deacetylase inhibitor to the individual. Such an administration upregulates the expression of p21 gene. Generally, the acute renal failure is caused by exposure to a contrast media dye or after failure of kidney, heart, lung and liver. Furthermore, representative examples of histone deacetylase inhibitor is phenylbutyrate, suberoylamide (SAHA), valproic acid, and trichostatin and their derivatives.

In yet another embodiment of the present invention, there is provided a method of treating a pathophysiological state in an individual, comprising: administering an histone deacetylase inhibitor to the individual, upregulating expression of a cell cycle regulator gene in the individual, and inducing cell cycle arrest, differentiation or apoptosis in damaged cells in the individual, thereby treating the pathophysiological state in the individual. Representative example of such cell cycle regulator gene is p21, although it may upregulate expression of other cell cyle regulators such as cyclin D and p27. Generally, the pathophysiological state in the individual is not limited to but includes lung cancer, ovarian cancer, testicular cancer or head and neck cancer. Representative exampled of histone deacetylase inhibitors are phenylbutyrate, suberoylamide, valproic acid, trichostatin and their derivatives.

The following definitions are given for the purpose of facilitating understanding of the inventions disclosed herein. Any terms not specifically defined should be interpreted according to the common meaning of the term in the art.

As used herein, the term “individual” shall refer to animals and humans.

Partial renal ablation leads to progressive renal insufficiency and is a model of chronic renal failure from diverse causes. Mice develop functional and morphologic characteristics of chronic renal failure after partial renal ablation including glomerular sclerosis, systemic hypertension and reduced glomerular filtration. However, litter-mates having a homozygous deletion of the gene for the cyclin-dependent kinase inhibitor, p21^{WAF1/CIP1}, do not develop chronic renal failure after ablation. The markedly different reactions of the p21(+/+) and p21(−/−) animals were not due to differences in glomerular number or degree of renal growth, but rather to the presence or absence of a normal p21 gene. While the reaction to the stress of renal ablation is both hyperplastic and hypertrophic in the presence of a functional p21 gene, the absence of the p21 gene may induce a more hyperplastic reaction since PCNA expression, a marker of cell-cycle progression, in the renal epithelium of the remnant kidney is more than five times greater in the p21(−/−) mice than in the p21(+/+) animals. As p21 is a potent inhibitor of the cell-cycle, p21 may regulate the balance between hyperplasia and hypertrophy following renal ablation. This change in response inhibits the development of chronic renal failure.

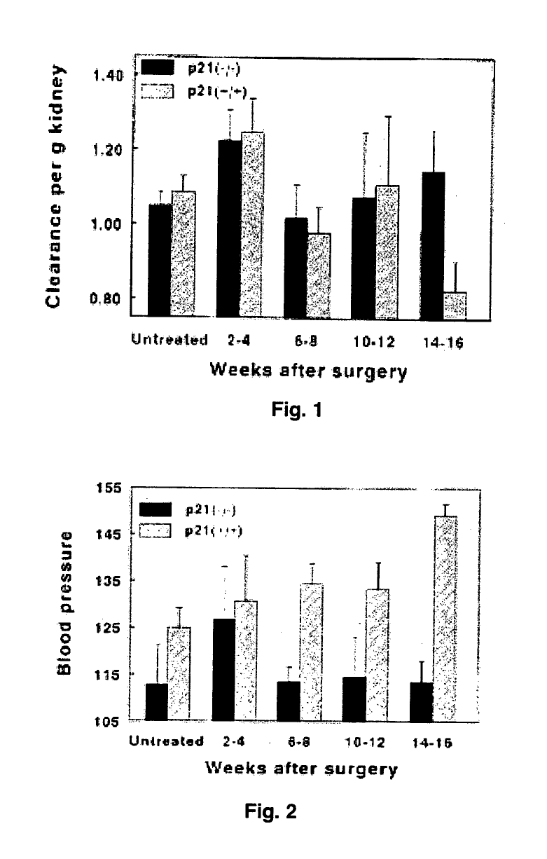

Mice lacking a p21 gene were resistant to the functional and morphologic consequences of partial renal ablation. Not only is the resistance manifested locally in the surgically impaired remnant organ, but it is also evident systemically in the lack of increased arterial pressure. This resistance may be due to several parameters that may be early determinants of the long-term outcome of renal ablation. Severe protein restriction can partially ameliorate the development of glomerulosclerosis after partial renal ablation (24). However, weight gains in the two groups of animals is not significantly different, and the p21(−/−) mice even experience slightly elevated gains, both relative and absolute. Reduced glomerular number may be an etiologic link in the progressive nature of renal disease (25,26). The p21(+/+) and (−/−) animals have similar numbers of glomeruli at the outset of the experiments (Table 1) and the degree of renal ablation is the same for each group. Thus the loss of renal excretory function is equally applied to both groups. The increase in glomerular filtration that occurs in response to renal ablation, also thought to be an early determinant of the progression (4), occurs to the same extent in the p21(−/−) animals as it does in the wild type (FIG. 1). Glomerular hypertrophy, which has an independent role in the progression of renal ablation models of experimental renal disease (27), occurs to the same extent in both groups as well (Table 2).

Taken together, this indicates that the p21 gene product plays a critical role in the functional and morphologic consequences subsequent to the stress of renal ablation, including the development of glomerular sclerosis and hypertension. Additionally, hypertension does not develop without the development of renal damage. This resistance may be critically linked to the prominent role the p21 protein (cdk) plays in regulating the cell cycle. The growth of the kidney following renal ablation is a consequence of hyperplasia and hypertrophy of the glomerular and epithelial compartments of the kidney (28,29). However, hypertrophy may be in the long term, a maladaptive response to the loss of functional renal tissue (4,27,30).

In the absence of the p21 gene the growth response of the kidney after partial ablation is relatively more hyperplastic than hypertrophic. Consistent with this notion is a greater than 5-fold increase in PCNA protein expression in p21(−/−) animals compared to the wild-type animals undergoing the response to renal ablation. By achieving growth after renal ablation by increasing the relative contribution of hyperplasia, the work load of the kidney is better accommodated. This assumes that when an organ accommodates increases in work by hypertrophy rather than hyperplasia, it is at a serious physiologic disadvantage and more likely to undergo regression of structure and function (31). A detailed description of the differences in the balance between hypertrophy and hyperplasia in the two groups of mice and, more specifically, the sites at which these differences are apparent would confirm this assumption. It is clear that p21 is a critical sensor of the stress of renal mass reduction. This model may be useful in identifying the mechanism of how this response to renal ablation is maladaptive. The studies also suggest that manipulation of p21 gene expression could be a target for the treatment of progressive renal failure.

Furthermore, the occurrence of acute renal failure during chemotherapy, following surgery, during infection, and after exposure to contrast media dye restricts the efficacy of the treatment and prevents cures. Additionally, its occurrence also increases mortality and in those that survive, it increases the cost of hospitalization. Thus, a therapy that enhances therapeutic efficacy of potentially nephrotoxic drugs while minimizing the toxicity of the drug would be of great benefit. Although histone deacetylase inhibitors upregulate the expression of p21 gene in normal and cancer cells, they exhibit differential affects in cancer cells as opposed to normal cells. Thus, it is possible that the upregulation of p21 by these inhibitors in cancer cells sensitized them to cancer chemotherapeutic agent cisplatin while the up-regulation of p21 in the normal kidney cells protected these normal cells from nephrotoxicity. Hence, the present invention relates to the relationship between cytotoxicity and the regulation of cell cycle through the p21 gene. The present invention also contemplates using this fundamental difference in regulation of the cell cycle to potential therapeutic benefit. This would improve the therapeutic potential of cisplatin, which remains the mainstay for the treatment of lung, ovarian, testicular and head and neck tumors, but is limited by its nephrotoxicity.

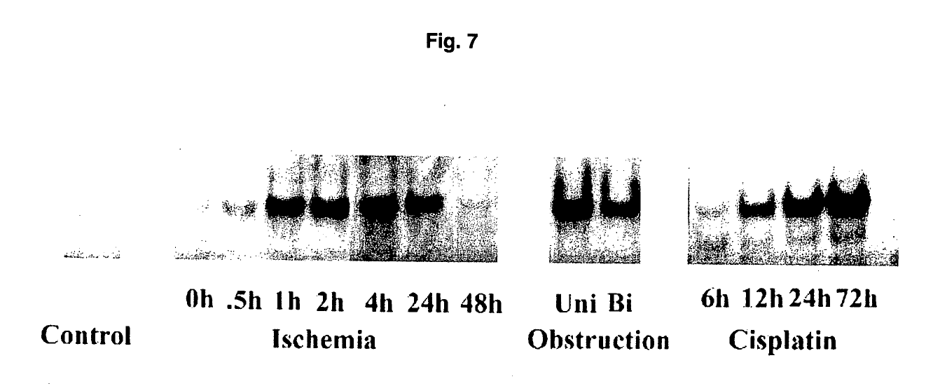

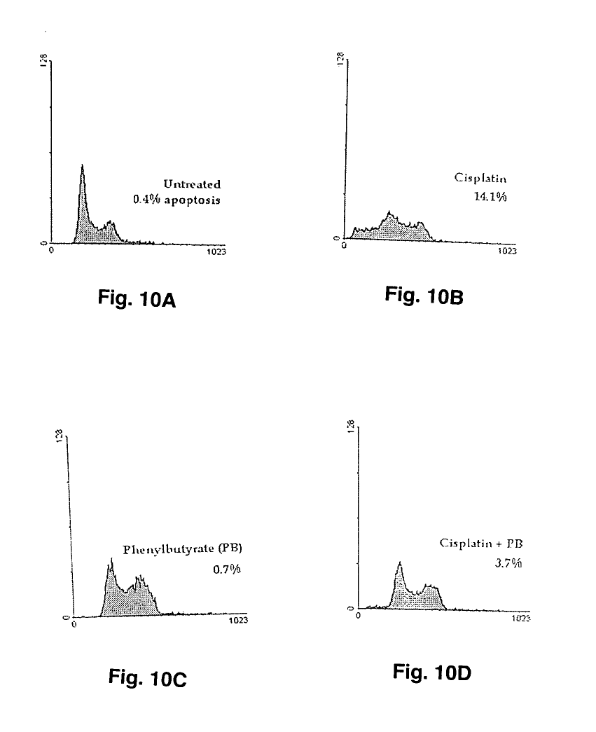

Currently, the use of histone deacetylase inhibitors in solid cancer or their use in combination with cisplatin is not known. The present invention demonstrated the protective effect of p21 gene when subjected to cisplatin-induced renal damage (FIGS. 7-9). Additionally, the present invention also demonstrated that renal cells exposed to cisplatin, but protected by an histone deacetylase inhibitor such as phenylbutyrate, which is a member of short chain fatty acid inhibitors of the histone deactylases, markedly protected cells from cisplatin-induced cell death (FIG. 10). Hence, this drug is used to inhibit the growth of tumor cells in vitro and in vivo. It is contemplated that HDAC inhibitors such as phenylbutyrate suberoylamide, valproic acid, and trichostatin and there derivatives might be used to diminish the side effects of all antineoplastic drugs including nephrotoxicity, mucositis, eneterocilitis and bone marrow suppression as well as limit radiation injury to normal tissue. It is further contemplated to use this apporach to upregulate the cyclin-dependent kinase inhibitor, p21 to prevent dye-induced acute renal failure, ischemic and the acute renal failure that occurs after kidney, heart, lung and liver failure.

The following examples are given for the purpose of illustrating various embodiments of the invention and are not meant to limit the present invention in any fashion.

### EXAMPLE 1

Methods and Materials: Animal Preparation

Mice (strain 129/Sv) carrying a deletion of a large portion of the p21 gene in which neither p21 mRNA nor p21 protein is expressed (14) were obtained from Dr. Philip Leder (Harvard Medical School, Cambridge, Mass.). Mice homozygous for the p21 deletion are selected from the offspring of heterozygous matings using Southern blotting of tail DNA as described (14). Wild-type p21(+/+) litter-mates are used as controls for a normal p21 gene. The animals are housed at the Animal Research Center at the University of Texas Medical Branch at Galveston. Food and water are supplied ad libitum. Body weights are determined at the start of the protocol, at the time of surgery, and at the time of sacrifice.

Renal ablation is created by two-step nephrectomy (15) using 6-8 week-old male mice. At the first stage of the procedure, the right kidney is decapsulated and the upper and lower poles are resected under anesthesia with Pentobarbital Sodium (50 mg/Kg) ip. Bleeding is prevented using a thrombin solution (3000 units/ml 0.9% NaCl). One week later, a total left nephrectomy is performed under anesthesia as described above. Renal function, kidney morphology, morphometry and mean arterial blood pressure are studied at various times thereafter.

Clearance and Direct Systolic Blood Pressure Measurements

Mice are anesthetized, as above, and placed on a heated surgical table to maintain body temperature between 37-38° C. Polyethylene catheters are placed in the trachea, bladder, both femoral arteries and left jugular vein. The mean arterial blood pressure is obtained via the left femoral artery using a strain-gauge transducer (Gould, Cleveland, Ohio). The animals are infused with 0.9% sodium chloride solution via the left external jugular vein at a rate of 0.5% body weight/hour using a constant infusion syringe pump (Model 355, Sage Instruments, Cambridge, Mass.). The infusion solution containe enough [Methoxy-³H]-inulin (American Radiolabeled Chemicals, St. Louis, Mo.) to deliver 10 μCi/hour. After a 60 minute equilibration period, urine is collected under mineral oil for three 30 minute clearance determinations. Blood is drawn in heparinized microhematocrit tubes from the right femoral artery at the beginning and end of the clearance period to determine hematocrit and [³H] activity. [³H] activity in urine and plasma is determined in a liquid scintillation counter (LKB Wallace 1211 RackBeta) and the GFR calculated.

Immunohistochemistry

Proliferating cell nuclear antigen (PCNA) is detected using a mouse monoclonal antibody (Santa Cruz Laboratory, Santa Cruz, Calif.) and the ABC Elite Vectastain Kit (Vector Laboratories, Inc., Burlingame, Calif.), according to manufacturers instructions.

In situ Hybridization

In situ localization of p21 mRNA on kidney sections was performed as previously described (5).

Quantitation of Glomerular Numbers Per Kidney

The number of glomeruli per kidney was determined by using the method described by MacKay et al (19).

Statistical Analysis

Results are presented as means±SE. Differences between means are evaluated using the Student's t-test for unpaired data. p<0.05 is considered statistically significant.

### EXAMPLE 2

Kidney Morphology and Morphometry

At the time of sacrifice, kidney remnants are freed from the surrounding tissues, weighed and cut in half, fixed in 4% neutral buffered formaldehyde, and processed for light microscopy by paraffin embedding. Sections (5 μm) are stained with hematoxylin-eosin, periodic-acid Schiff (PAS) or trichrome.

Morphological Studies

Three to five animals at various time points are used for morphological studies. Using PAS-stained sections, at least 300 glomeruli are evaluated by light microscopy. The percentage of each glomerulus exhibiting mesangial expansion or glomerulosclerosis was determined by point counting (4) at ×400 using an eyepiece reticle (SO75963, Nikon Inc.) Focal glomerulosclerosis is graded as to percent of glomerular area sclerotic using the following criteria: minimal (1-25%), moderate (26-50%) and severe (51-84%). When ˜85% of glomerular area is sclerotic, the glomerulus is classified as globally sclerotic.

Glomerular Morphometry

To determine glomerular hypertrophy mean glomerular volume (MGV, μm³) is measured based on point counting (16-18) according to the following formula:
MGV=1.25[(antilog (log P)/n)k²]^{3/2}, where
P=number of points falling on each glomerular tuft profile;
k=distance between the points in micrometers; and
n=number of glomeruli counted.

Glomeruli showing global sclerosis were excluded.

Glomerular Filtration Rate (GFR)
C_{Inulin}(ml/min)=U/P[³H]×Vu(ml/min)
Percent (%) Nephrectomy and Hypertrophy

$$\% nephrectomy = \frac{{RK}_{REMOVED} + {LK}_{ADJ}}{2 \times {LK}_{ADJ}} \times 100 , where$$

RK_{REMOVED} is the amount (mg) of the right kidney removed in the first operation and LK_{ADJ} is the weight (mg) of the left kidney removed in the second operation 7 days later, adjusted for hypertrophy between the first and second operation. The adjustment is calculated by multiplying the weight of the left kidney at the time of removal by the average kidney weight per body weight of untreated animals divided by the average kidney weight per body weight of day 7 left kidneys.
Percent (%) Hypertrophy

$$\% hypertrophy = \frac{{RK}_{FINAL} - {RK}_{INTACT}}{{RK}_{INTACT}} \times 100 , where$$

RK_{FINAL} was the weight (mg) of right kidney at sacrifice; and RK_{INTACT} is LK_{ADJ}−RK_{REMOVED}.

### EXAMPLE 3

Body Weight and Renal Parameters before Ablation

Body weight, kidney weight, glomerular number and volume, and renal function in untreated p21(+/+) and (−/−) mice are given in Table 1. There are no phenotypic differences between the two groups of mice, although the untreated p21(−/−) animals are about 15% (p<0.001) larger than those in the p21(+/+) group. Size increases have also been reported in mice lacking the p27 cdk inhibitor genes (20-23). However, neither kidney weight per gram body weight, total glomerular number, nor mean glomerular volume are different between the two genotypes. Similarly, the two-kidney glomerular filtration rate (GFR, expressed as Cᵢₙᵤₗᵢₙ) of the untreated animals is not different.

TABLE 1
Physical Parameters in Untreated Mice

| | | | | Mean Glomerular | |
|---|---|---|---|---|---|
| | Body Weight | Kidney Wt. | Number of | Vol. × 10⁻³ | Cᵢₙᵤₗᵢₙ |
| | (gm) | (mg/gm body weight) | Glomeruli/Kidney | (μm³) | ml/min |
| p21 (+/+) | 24.35 ± 2.68 | 5.938 ± 0.66 | 12583 ± 681 | 1.92 ± 0.58 | 1.09 ± 0.07 |
| p21 (−/−) | 28.47 ± 4.18 | 5.720 ± 0.61 | 12091 ± 555 | 1.74 ± 0.1 | 1.05 ± 0.08 |
| p value | p < 0.001 | NS | NS | NS | NS |

Values are means ± standard deviation.
NS = not significant

### EXAMPLE 4

Body Weight and Renal Parameters after Ablation

Weight gain in renal ablated mice throughout the 14-16 week period of observation was not significantly different between the two groups, either in absolute terms (2.3±0.8 g vs 4.3±1.1 g; +/+ vs −/− groups, respectively; n=11 in each group) or relative to initial body weight (10.2±3.5% vs 15.9±4.3%; +/+ vs −/− groups, respectively). The degree of renal ablation was determined for each genotype. Approximately ⅔ of the normal renal mass was removed after the 2 operations and there is no significant difference between the groups. The percent nephrectomy in the p21(+/+) and p21(−/−) groups was 68.8±3.6% and 68.3±3.1% (p=0.619), respectively. Furthermore, the degree of hypertrophy and the mean glomerular volume after ablation (Table 2) was not significantly different between the groups.

TABLE 2
Percent hypertrophy and mean glomerular volumes after renal ablation

| | | | | MGV × 10⁵ (μm³) | | |
|---|---|---|---|---|---|---|
| | Hypertrophy (%) | | | p21 | p21 | |
| Weeks | p21 | p21 | t-test | (+/+) | (−/−) | t-test |
| Control | NA | NA | NA | 1.92 ± 0.58 | 1.74 ± 0.14 | NS |
| 2-4 | 66.9 ± 28.6 | 83.9 ± 85.7 | NS | 1.93 ± 0.32 | 2.58 ± 0.71 | NS |
| 6-8 | 86.8 ± 41.5 | 138.5 ± 55.8 | NS | 2.71 ± 0.33 | 2.99 ± 0.34 | NS |
| 10-12 | 137.1 ± 88.7 | 141.4 ± 31.3 | NS | 3.52 ± 0.21 | 3.34 ± 0.37 | NS |
| 14-16 | 145.0 ± 37.0 | 135.9 ± 42.1 | NS | 2.87 ± 0.50 | 3.17 ± 0.38 | NS |

Values are means ± standard deviation.
NS = not significant;
NA = not applicable.

### EXAMPLE 5

Renal Function following Ablation

Glomerular filtration rate increased to the same extent 2 to 4 weeks after ablation in both groups. Glomerular filtration rate was similar in both groups until the 14^{th}-16^{th} week after ablation when it falls in the wild-type animals but remains unchanged from previous values in the p21(_/_) group. The glomerular filtration rate at this time point was significantly different between the two groups (p<0.05) (FIG. 1).

### EXAMPLE 6

Mean Arterial Pressure

Mean arterial pressure is not significantly different between the untreated groups of animals. Following partial renal ablation, arterial pressure increases initially in both groups of animals and increases further in the p21(+/+) mice so that by the 14^{th}-16^{th} week the average mean systolic pressure reaches 150.7±6.7 mm Hg (mean±SD). By contrast, mean systolic blood pressure in the p21(−/−) mice returns toward normal and remains there throughout the 16-week period of observation (113.8±17.7 after 16 weeks versus 112.8±16.7 in untreated mice) (FIG. 2).

### EXAMPLE 7

Morphology

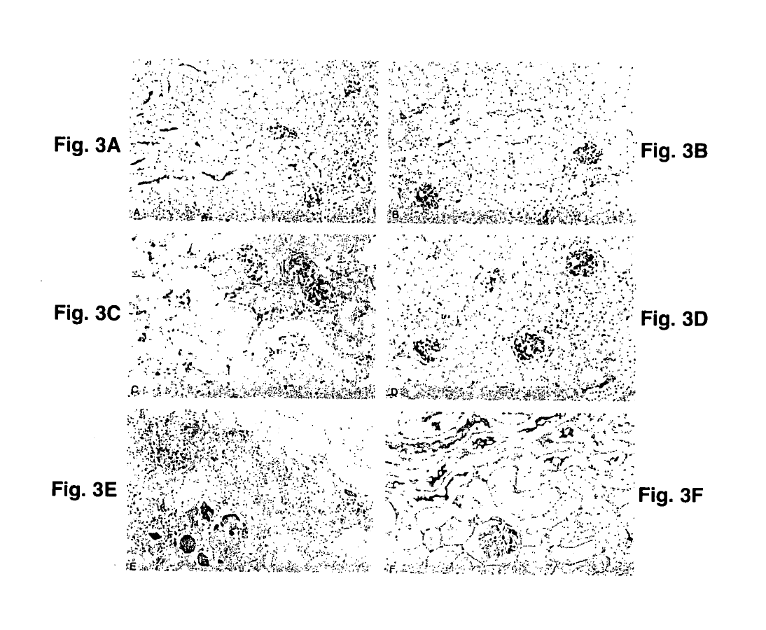

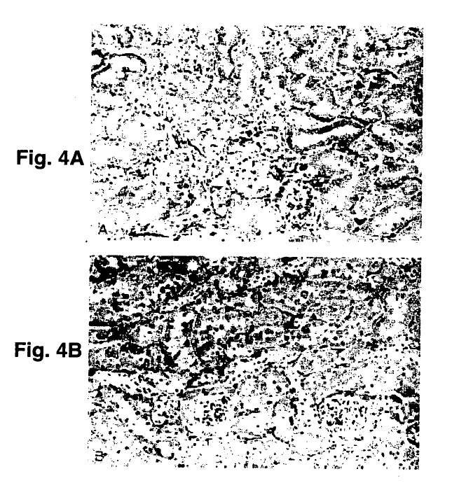

Light microscopic study reveals a marked difference of histologic changes between the two groups of mice. Representative micrographs are given in FIGS. 3 and 4; the changes are quantified in Table 3. Kidney sections from untreated mice were morphologically indistinguishable (FIGS. 3A, 3B). Mesangial expansion and mild focal glomerulosclerosis was observed in about 70% of glomeruli in the p21(+/+) mice 4 weeks after ablation (Table 3). Beginning at 6 to 8 weeks these mice developed severe focal and global glomerulosclerosis (FIG. 3C [cf FIG. 3D], FIG. 4A, Table 3). All of the p21(+/+) mice studied developed glomerulosclerosis accompanied by interstitial fibrosis and round cell infiltration by 14-16 weeks post ablation (FIG. 3E, Table 3). In contrast, p21(−/−) mice never developed glomeruloscierosis nor interstitial changes even 26 weeks after renal ablation (FIG. 3F, FIG. 4B) although mesangial expansion was seen occasionally.

The percentages of glomerulosclerosis in the p21(+/+) mice at various times after ablation are quantified in Table 3. It can be seen that they developed a progressive increase in glomerular sclerosis. The p21(−/−) mice do not develop glomerulosclerosis throughout the period of observation and were omitted from the table.

TABLE 3
Glomerulosclerosis in p21 (+/+) Mice

| Weeks | | None | Minimal | Moderate | Severe | Global |
|---|---|---|---|---|---|---|
| 4 | W | 30.7 ± 0.9 | 65.8 ± 1.6 | 2.8 ± 1.7 | 0.8 ± 0.7 | 0 |
| 6-8 | W | 27.8 ± 5.1 | 41.0 ± 2.7 | 22.2 ± 3.5 | 4.9 ± 1.8 | 4.2 ± 3.5 |
| 10-12 | W | 15.3 ± 3.2 | 38.2 ± 7.8 | 25.2 ± 3.5 | 10.2 ± 3.3 | 11.1 ± 6.8 |
| 14-16 | W | 23.6 ± 2.5 | 22.4 ± 4.4 | 36.5 ± 4.1 | 9.5 ± 1.9 | 8.1 ± 3.9 |

Percent glomeruli in each category (±standard error) as defined above.

### EXAMPLE 8

Expression of p21 in the Remnant Kidney

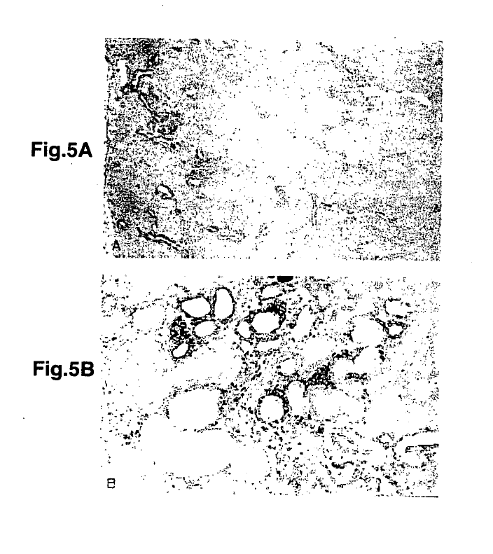

In situ hybridization for p21 mRNA identifies the cells of the cortical thick ascending limbs and distal convoluted tubules as the principal site of p21 expression 4 weeks following ablation (FIG. 5A). At later times, it was also expressed in the epithelium of tubules (primarily dilated and collapsed) and glomeruli within or adjacent to sclerotic areas of the remnant kidney (FIG. 5B).

### EXAMPLE 9

Cell Cycle Analysis

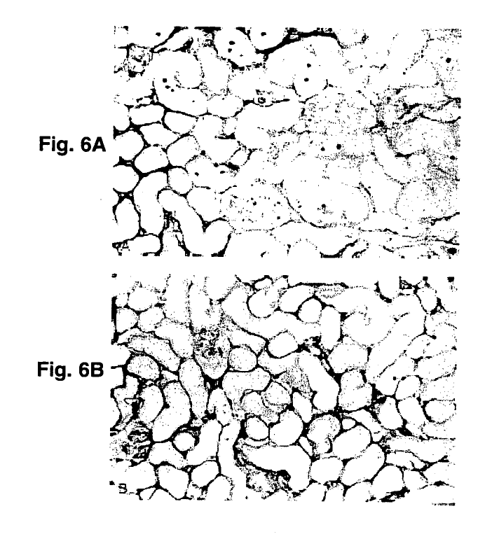

Nuclear PCNA, a marker for cells in the S phase of the cell cycle is found in many cells of the remnant kidney in the p21(−/−) mice 2 weeks after surgery (FIG. 6A). The positive nuclei are primarily localized in the proximal convoluted tubules and occasionally in the glomeruli and distal convoluted tubules. By contrast, few cell nuclei are stained in the p21(+/+) remnant kidney (FIG. 6B). This difference in PCNA staining is quantified in nuclei from p21 (−/−) mice (18.64±0.73 per mm²) and p21(+/+) mice (3.50±0.65 per mm²) and is highly significant (p=0.00006). At later time points, PCNA is greatly diminished in both animals (data not shown).

### EXAMPLE 10

Protective Effect of p21 Gene on Renal Function

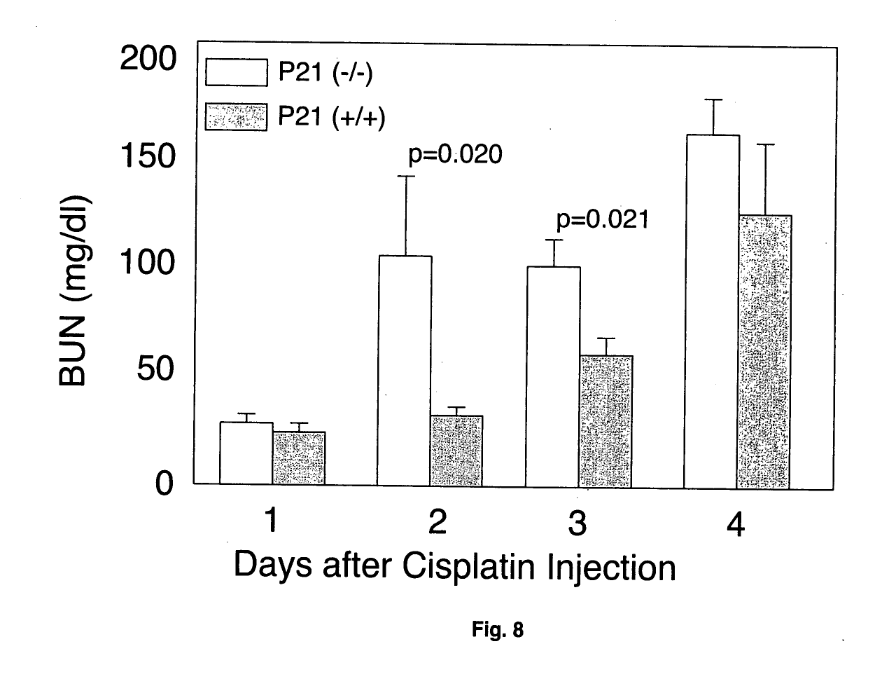

The present invention examined the effect of p21 in protecting the renal function from cisplatin induced damage. An increase in renal p21 expression was observed after ischemia-reperfusion, urinary tract obstruction and cisplatin administration (FIG. 7). Further, to assess the significance of p21 in renal function, blood urea nitrogen was measured in mice with and without p21 gene subsequent to cisplatin administration. It was observed that the blood urea nitrogen for mice lacking the p21 gene was retained to a higher degree than the mice with p21 gene (FIG. 8). This demonstrated a greater renal failure in the mice without p21 gene and thus, indicated the importance of p21 gene in preventing renal failure.

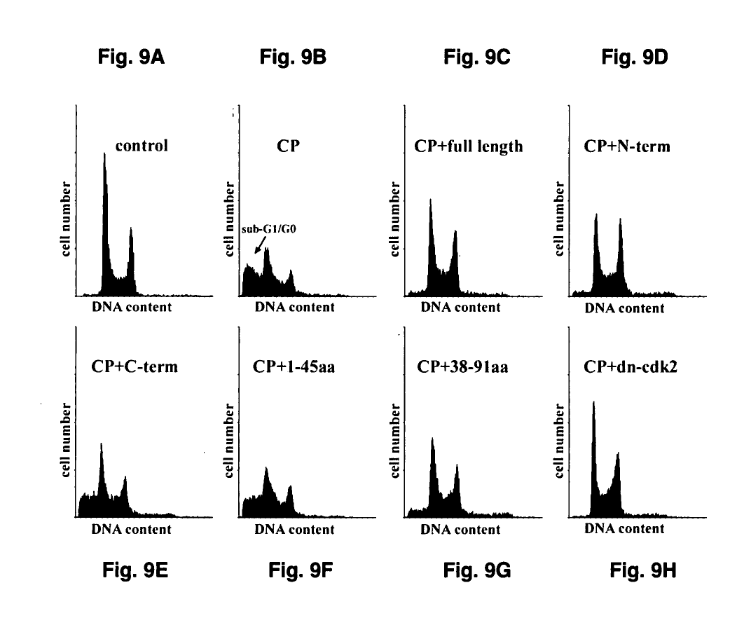

Additionally, the effect of p21 gene on cisplatin-induced apoptosis in renal proximal tubule cells was assessed. These cells were incubated without any treatment, with cisplatin alone, cisplatin and full length p21 protein, cisplatin and N-terminal portion of the p21 protein, Cisplatin and C-terminal portion of the p21 protein, cisplatin and 1-45 amino acids of the p21 protein, cisplatin and 38-91 amino acids of the p21 protein or cisplatin and dominant negative cdk2 and the DNA content was measured. It was observed that the N-terminal portion of the p21 protein was the protective part of the molecule and protected the cells from cisplatin induced apoptosis (FIG. 9D). It was also observed that inhibition of cdk2, which is the principal target of p21 also protected the cells from damage. In summary, the present invention demonstrated that p21 protected the renal cells from cisplatin-induced renal damage.

### EXAMPLE 11

Cytoprotective Effect of Phenylbutryate on Renal Cells

Next, the cytoprotective effect of histone deacetylase inhibitors such as phenylbutyrate on renal cells was examined. Cells were control cells, or exposed to cisplatin (50 uM) alone, to phenylbutyrate (100 uM) or to phenybutyrate and cisplatin. It was observed that cells exposed to cisplatin alone showed fragmented apototic distribution of DNA (FIG. 10B). In distinct contrast, cell exposed to ciplatin and phenylbutyrate showed a normal distribution of DNA (FIG. 10D). Thus, it indicated that renal cells exposed to cisplatin were protected from apoptosis by prior exposure to phenylbutyrate, a member of the short chain fatty acid inhibitors of the histone deactylases.

### EXAMPLE 12

Cytoprotective Effect of HDAC Inhibitors on Lung Cancer Cells

The cytoprotective effect of an histone deacetylase inhibitor on lung cancer cells is examined. In order to do so, the NSCLC cell line CRL 5895 (insensitive to platinum), CRL 5897 (partial response to platinum), SCLC HCC4001 (cisplatin-sensitive cell line) and HCC4004 (cisplatin-resistant cell line) are used. These cell lines and proximal tubule cells are incubated with cisplatin (10-100 uM) with and without a histone deacetylase inhibitor. Cell death is measured at 24 hours after exposure by annexin-binding and survival curves. The histone deacetylase inhibitor shifted the survival to the left in cancer cells (i.e. sensitize them) while shifting the survival to the right (protecting them) in renal cells. The dependence of these shifts on the presence and absence of the p21 gene is also evaluated in p21 KO tumor cells and renal cells derived from p21 KO mice. Taken together, the results demonstrate that histone deacetylase inhibitors sensitize the lung cancer cells to cisplatin and protect renal epithelial cells from cisplatin and that these effects are p21-mediated.

### EXAMPLE 13

Protective Effect of an HDAC Inhibitor against Cisplatin-Induced Renal Failure in vivo

The protective effect of a histone deacetylase inhibitor against cisplatin-induced renal failure and the dependence of this protective effect on the presence of a normal p21 gene in vivo is examined. In order to do so, wild type (WT) and p21 KO mice are exposed to a nephrotoxic dose of cisplatin (50 ug/kg) in the presence and absence of an histone deacetylase inhibitor. The extent of renal damage is assessed by measurements of BUN and Creatinine as well as quantitative assessment of tubule damage 3, 7 and 14 days after treatment with cisplatin. Lower azotemia and reduced proximal tubule damage is observed in presence of the inhibitor in wild type animals while this protective effect is abrogated in the absence of p21 gene.

### EXAMPLE 14

Protective Effect of an HDAC Inhibitor in Tumor-Bearing Nude Mice

Further, whether tumor-bearing nude mice respond better to cisplatin when combined with an histone deacetylase inhibitor and with less nephrotoxicity than the mice treated with cisplatin alone is examined. In order to do so, lung cancer bearing nude mice are exposed to cisplatin in presence and absence of an histone deacetylase inhibitor. The extent of tumor regression and renal damage is meausured. The tumors on the nude mice regresses more with less nephrotoxicity in mice receiving the histone deacetylase inhibitor than the ones not receiving the inhibitor.

The following references are cited herein:
1. Chanutin, A. & Ferris, E. B., Jr. (1932) Arch. Intern. Med. 49, 767-787.
2. Morrison, A. B. (1962) Lab Invest. 11, 321-332.
3. Faraj, A. H. & Morley, A. R. (1992) APMIS 100, 1097-1105.
4. Daniels, B. S. & Hostetter, T. H. (1990) Am. J. Physiol. 258, F1409-F1416.
5. Megyesi, et al., (1996) Am. J. Physiol. 271, F1211-F1216.
6. Gorospe, et al., (1996) Mol. Cell. Differ. 4, 47-65.
7. El-Deiry, et al., (1993) Cell 75, 817-825.
8. Xiong, et al., (1992) Cell 71, 505-514.
9. Harper, et al., (1993) Cell 75, 805-816.
10. Noda, et al., (1994) Exp. Cell Res. 211, 90-98.
11. Steinman, et al., (1994) Oncogene 9, 3389-3396.
12. Jiang et al., (1994) Oncogene 9, 3397-3406.
13. Megyesi, et al., (1997) J. Clin. Invest. 101, 777-782.
14. Deng, et al., (1995) Cell 82, 675-684.
15. Hamamori, et al., (1995) J. Clin. Invest. 95, 1808-1813.
16. Nath, et al., (1990) J. Clin. Invest. 86, 1179-1192.
17. Hirose, et al., (1982) Kidney Int. 21, 689-695.
18. Bilous, et al., (1989) Diabetes 38, 1142-1147.
19. MacKay, et al., (1987) Kidney Int. 32, 827-837.
20. Nakayama, et al., (1996) Cell 85, 707-720.
21. Kiyokawa, et al., (1996) Cell 85, 721-732.
22. Fero, et al., (1996) Cell 85, 733-744.
23. Franklin, et al., (1998) Genes Dev. 12, 2899-2911.
24. Brenner, et al., (1982) N. Engl. J. Med. 307, 652-660.
25. Brenner, B. M., Garcia & Anderson (1988) Am. J. Hypertens. 1, 335-347.
26. Fetterman, G. H. & Habib, R. (1969) Am. J. Clin. Path. 52, 199-207.
27. Yoshida, et al., (1989) Kidney Int. 35, 654-660.
28. Terzi, et al., (1995) Am. J. Physiol. 268, F793-F801.
29. Floege, et al., (1992) Kidney Int. 41, 297-309.
30. Fries, et al., (1989) Lab. Invest. 60, 205-218.
31. Goss, R. J. (1966) Science 153, 1615-1620.
32. Chen, et al., (2003) Current Cancer Drug Targets. 3, 219-236.
33. Richon, et al., (2000) PNAS. 97, 10014-10019.
34. Megyesi, et al., (2002) 283(4): F810-816.

Any patents or publications mentioned in this specification are indicative of the levels of those skilled in the art to which the invention pertains. These patents and publications are herein incorporated by reference to the same extent as if each individual publication was specifically and individually incorporated by reference. One skilled in the art will readily appreciate that the present invention is well adapted to carry out the objects and obtain the advantages mentioned, as well as those inherent therein. It will be apparent to those skilled in the art that various modifications and variations can be made in practicing the present invention without departing from the spirit or scope of the invention. Changes and other uses will occur to those skilled in the art which are encompassed within the spirit of the invention as defined by the claims.

1. A method for treating or preventing a pathophysiological state of an organ in an individual wherein said state is characterized by an undesirable level of cyclin-dependent kinase inhibitor activity in said organ, comprising the step of regulating the expression of the p21 gene in said organ of said individual.

2. The method of claim 1, wherein said organ is selected from the group consisting of kidney, heart, liver, and lung.

3. The method of claim 1, wherein said pathophysiological state is selected from the group consisting of renal fibrosis, glomerulosclerosis, reduced filtration rates, hypertension, and organ transplantation rejection.

4. The method of claim 1, wherein the regulation of the expression of the p21 gene results in the reduction or elimination of the expression of the p21 gene.

5. The method of claim 4, wherein the reduction or elimination of the expression of the p21 gene is performed by a technique selected from the group consisting of drug therapy, and genetic manipulation.

6. A method for treating or preventing chronic progressive renal failure in an individual, comprising the step of regulating the expression of the p21 gene in one or both kidneys of said individual wherein said regulation of the p21 gene results in the manipulation of cyclin-dependent kinase inhibitor activity in one or both kidneys.

7. The method of claim 6, wherein the regulation of the expression of the p21 gene results in the reduction or elimination of the expression of the p21 gene.

8. The method of claim 7, wherein the reduction or elimination of the expression of the p21 gene is performed by a technique selected from the group consisting of drug therapy, and genetic manipulation.

9. A method of lowering the rate of long-term rejection of a transplanted organ in an individual comprising the step of transplanting into said individual the organ from a donor wherein the p21 gene in said organ is not expressed.

10. The method of claim 9, wherein said organ is selected from the group consisting of kidney, heart, liver, and lung.

11. A method of enhancing therapeutic potential of a chemotherapeutic drug in an individual being treated for a pathophysiological state, comprising:
administering an histone deacetylase inhibitor to said individual; and
sensitizing target cells to the chemotherapeutic drug and protecting normal cells from the drug-induced cytotoxicity, thereby enhancing the therapeutic potential of the chemotherapeutic drug in the individual.

12. The method of claim 11, wherein said administration upregulates the expression of p21 gene.

13. The method of claim 11, wherein the drug-induced cytotoxicity is nephrotoxicity, mucositis, enterocilitis or bone marrow suppression.

14. The method of claim 11, wherein said histone deacetylase inhibitor is phenylbutyrate, suberoylamide, valproic acid, trichostatin and derivatives of said inhibitors.

15. The method of claim 11, wherein said chemotherapeutic drug is cisplatin, all-trans retinoic acid, gemcitabine and amphotericin.

16. The method of claim 11, wherein said pathophysiological state is lung cancer, ovarian cancer, testicular cancer or head and neck cancer.

17. A method of treating or preventing acute renal failure in an individual, comprising the step of administering an histone deacetylase inhibitor to said individual.

18. The method of claim 17, wherein said administration upregulates the expression of p21 gene.

19. The method of claim 17, wherein said acute renal failure is caused by exposure to a contrast media dye or after failure of kidney, heart, lung or liver.

20. The method of claim 17, wherein the histone deacetylase inhibitor is phenylbutyrate, suberoylamide (SAHA), valproic acid, trichostatin and derivatives of said inhibitors.

21. A method of treating a pathophysiological state in an individual, comprising:
administering an histone deacetylase inhibitor to the individual;
upregulating expression of a cell cycle regulator gene in the individual:and
inducing cell cycle arrest, differentiation or apoptosis in damaged cells in the individual, thereby treating the pathophysiological state in the individual.

22. The method of claim 21, wherein said cell cycle regulator gene is p21.

23. The method of claim 21, wherein said pathophysiological state is lung cancer, ovarian cancer, testicular cancer or head and neck cancer.

24. The method of claim 21, wherein the histone deacetylase inhibitor is phenylbutyrate, suberoylamide, valproic acid, trichostatin and derivatives of said inhibitors
